(19) United States
(12) Patent Application Publication
(10) Pub. No.: US 2003/0149292 A1
(43) Pub. Date: Aug. 7, 2003

(54) Method for preparing alkyl nitrites

(76) Inventors: Philippe Karrer, Zillisheim (FR); Sebastien Balzano, Lutterbach (FR)

Correspondence Address: BURNS DOANE SWECKER & MATHIS L L P, POST OFFICE BOX 1404, ALEXANDRIA, VA, 22313-1404, US

(21) Appl. No.: 10/240,319

(22) PCT Filed: Apr. 3, 2001
(86) PCT No.: PCT/FR01/00999

(30) Foreign Application Priority Data
Apr. 3, 2000 (FR) 00/04231

(51) Int. Cl. C07C 203/00

(52) U.S. Cl. 558/486

(57) ABSTRACT

The invention concerns a method for preparing alkyl nitrites of formula (I) R ONO (I) ONO wherein R represents a C₁-C₂₀, advantageously C₂-C₁₀ linear or branched alkyl group. The invention is characterised in that it consists in gradually and continuously adding in an aqueous medium, an alcohol of formula (II) R OH, R being as defined above, a nitrite of formula (III) M NO₂, wherein M represents a metal cation, and a strong acid, so as to form continuously said alkyl nitrite, and in continuously drawing off said alkyl nitrite thus formed from the reaction medium.

The invention relates to the continuous synthesis of alkyl nitrites by reacting an alcohol with an inorganic nitrite in an acidic medium.

Alkyl nitrites are used, inter alia, in the pharmaceutical field as vasodilators, in the field of colorants as nitrosating reagents, in the field of polymers as reaction catalysts or as additives for fuels.

Alkyl nitrites may be obtained by several routes.

A first route, described by Matsunaga et al. [Kogyo Kayaku (1980), 41(1), 3-7), consists in reacting an alkyl halide with an inorganic nitrite, in particular sodium nitrite.

A second route consists in reacting an aliphatic alcohol with a nitrosating reagent.

The nitrosating reagent may be nitrosyl chloride, as described by Hiroshi et al. [J. Chem. Soc. Perkin Trans. I; (1992), 14, 1837-1842].

The nitrosating reagent may also be nitrous acid, which is generated in situ by addition, to the aliphatic alcohol, of an inorganic nitrite in the presence of a strong acid.

This route of synthesis is described in particular by Hiroshi et al. (cited above), T. J. Blacklock [J. Org. Chem. (1989), 54(16), 3907-3913] and S. Glover [S. Afr. J. Chem.; (1981) 34(4), 96-100].

All the methods for the synthesis of alkyl nitrites from an aliphatic alcohol and from an inorganic nitrite used up until now are based on batch operations with, as main drawbacks, a low productivity and an increased risk of degradation of the product linked to its long residence time in the reaction medium.

The work by the inventors who are the authors of the present invention has led to a continuous process which makes it possible, in a simple manner and economically, to prepare alkyl nitrites by reacting nitrous acid, formed in situ, with an aliphatic alcohol, with a high productivity with respect to the final product.

The subject of the present invention is a method for preparing alkyl nitrites of formula I:

R—ONO (I)

in which R represents a C₁-C₂₀, advantageously C₂-C₁₀, linear or branched alkyl group, characterized in that there are gradually and continuously added to an aqueous medium

an alcohol of formula II:

R OH (II)

R being as defined above,

a nitrite of formula III:

M NO₂ (III)

in which M represents an alkali or alkaline-earth metal cation, and

a strong acid, so as to continuously form said alkyl nitrite, and in that said alkyl nitrite thus formed is continuously drawn off from the reaction medium.

Advantageously, R represents a C₂-C₅ alkyl group.

M preferably represents sodium or potassium.

The inorganic acid is advantageously a strong acid. The expression strong acid is understood to mean an acid having a pKa of less than 3.3.

As strong acid, there may be mentioned hydrochloric acid or sulfuric acid.

Preferably, the concentration of the strong acid solution supplied to the reaction medium is at least 35%.

The reaction is carried out at a temperature of between 0° C. and 15° C., advantageously between 0° C. and 5° C.

The reagents are simultaneously and continuously added so that the reactive species are present in the reaction medium at low instantaneous concentrations.

Preferably, care is taken that the concentrations of reactive species in the aqueous reaction medium do not exceed 6.1-6.2, 4-5 and 6.3-7.5 μmol/1 for the aliphatic alcohol, the inorganic nitrite and the strong acid respectively.

Advantageously, the molar ratio of the strong acid to the alcohol is from 1 to 1.5,and preferably from 1 to 1.1, and the molar ratio of the strong acid to the nitrite is not greater than 1.

These conditions should be met during the progress of the reaction.

However, at the beginning of the reaction, the molar ratios indicated above may exceed the limits indicated.

According to a first variant of the embodiment of the invention:

a) a stock solution is prepared comprising an aqueous medium, a fraction of the compound of general formula II and a fraction of the compound of general formula III,

b) said strong acid, the remaining quantity of said alcohol of general formula II and the remaining quantity of the metal nitrite of general formula III are gradually and continuously added to this stock solution.

It is also possible to use correctly calibrated metering pumps, which allows the omission of step a).

According to a second variant of the method of the invention, the product of general formula I formed is continuously drawn off from the reaction medium and the product formed is continuously dried.

This may be carried out on a molecular sieve or potassium hydroxide column.

In this manner, the low residence time of the product formed in the acidic medium reduces the risks of degradation of the nitrous ester.

The drying of the final product also makes it possible to increase the stability of the product formed over time by removing the water and the traces of acidity which are prescribed in the nitrous ester and which are responsible for the decomposition of the product.

The examples below illustrate the invention.

### EXAMPLES

### Example 1

### Preparation of N-butyl Nitrite

Into a one liter cylindrical reactor, provided with stirring, with a condenser and with three dropping funnels, are loaded under nitrogen, as a stock solution:

92 g of water containing 5% technical sodium chloride.

The three dropping funnels are respectively loaded with:

185.3 g of n-butanol

461.4 g of aqueous solution containing 40% of sodium nitrite (1.07 eq)

273.8 g of 35% hydrochloric acid (1.05 eq). 10% of the n-butanol and 10% of the aqueous NaNO₂ solution are added to the stock of water beforehand in order to start with a slight excess of these reagents and thus avoid losing NaNO₂ in the form of nitrous acid. The potential nitrous vapors are trapped in a round-bottomed flask containing 400 g of water and 200 g of 50% potassium hydroxide.

The stock of water containing the salt is cooled to 2±2° C. and the 3 reagents are simultaneously added at this temperature over about 1 hour (very exothermic reaction). The medium is maintained at this temperature for 5 minutes.

The bottom aqueous phase is siphoned and the top organic phase is washed with:

92.6 g of water.

The organic phase is dried by passage over a potassium hydroxide column.

246.6 g of dry n-butyl nitrite are thus obtained, that is a yield of 95.6% titrating 97-98% purity (GC n-butanol content: 1.7-1.9%).

### Example 2

### Preparation of Isoamyl Nitrite

Into a one liter cylindrical reactor, provided with stirring, with a condenser and with three dropping funnels, are loaded under nitrogen, as a stock solution:

77 g of water containing 5% technical sodium chloride.

The three dropping funnels are respectively loaded with:

176.2 g (2 mol) of isoamyl alcohol

370 g of aqueous solution containing 40% of sodium nitrite (1.07 eq)

219.3 g of 35% hydrochloric acid (1.05 eq). 10% of the isoamyl alcohol and 10% of the aqueous NaNO₂ solution are added to the stock of water beforehand in order to start with a slight excess of these reagents and thus avoid losing NaNO₂ in the form of nitrous acid.

The potential nitrous vapors are trapped in a round-bottomed flask containing a potassium hydroxide solution.

The stock of water containing the salt is cooled to 2±2° C. and the 3 reagents are simultaneously added at this temperature over about 1 hour (exothermic reaction, cooling with brine to −12° C.).

The top organic phase is washed with:

2×60 g of water.

The organic phase (226 g) is then dried by passage over a molecular sieve. 226 g of dry isoamyl nitrite are thus obtained, that is a yield of 96.4% titrating 98% purity.

1. A method for preparing alkyl nitrites of formula I:
R—ONO (I)
in which R represents a C₁-C₂₀, advantageously C₂-C₁₀, linear or branched alkyl group, characterized in that there are gradually and continuously added to an aqueous medium
an alcohol of formula II
R OH (II)
R being as defined above,
a nitrite of formula III
M NO₂ (III)
in which m represents a metal cation, preferably an alkali or alkaline-earth metal cation, and
a strong acid, preferably inorganic, so as to continuously form said alkyl nitrite, and in that said alkyl nitrite thus formed is continuously drawn off from the reaction medium:

2. The method as claimed in claim 1, characterized in that the alkyl nitrite drawn off from the reaction medium is dried.

3. The method as claimed in claim 1, characterized in that during the reaction, the concentration of alcohol of formula II is not greater than 6.1-6.2 μmol/l.

4. The method as claimed in claim 1, characterized in that during the reaction, the concentration of nitrite of formula III is not greater than 4-5 μmol/l.

5. The method as claimed in claim 1, characterized in that during the reaction, the concentration of inorganic acid is not greater than 6.3-7.5 μmol/l.

6. The method as claimed in claim 1, characterized in that the molar ratio of the acid to the alcohol is from 1 to 1.5, preferably 1 to 1.1.

7. The method as claimed in claim 1, characterized in that the molar ratio of the acid to the nitrite is not greater than 1.

8. The method as claimed in claim 1, characterized in that
a) a stock solution is prepared comprising an aqueous medium, a fraction of the compound of general formula II and a fraction of the compound of general formula III,
b) said acid, the remaining quantity of said alcohol of general formula II and the remaining quantity of the metal nitrite of general formula III are gradually and continuously added to this stock solution.

9. The method as claimed in claim 1, characterized in that the product formed of general formula I is continuously dried over a molecular sieve or potassium hydroxide column.

10. The method as claimed in any one of the preceding claims, characterized in that R represents the n-butyl or isoamyl group.

11. The method as claimed in any one of the preceding claims, characterized in that the strong acid is hydrochloric or sulfuric acid.

12. The method as claimed in any one of the preceding claims, characterized in that the alkali metal nitrite is sodium or potassium nitrite.
